(19) United States
(12) Patent Application Publication
(10) Pub. No.: US 2025/0018118 A1
(43) Pub. Date: Jan. 16, 2025

(54) A COVER STRUCTURE FOR A MEDICAMENT DELIVERY DEVICE

(71) Applicant: SHL Medical AG, Zug (CH)

(72) Inventors: Daniel Scott, Deerfield Beach, FL (US); Nurettin Ali, Deerfield Beach, FL (US)

(21) Appl. No.: 18/704,331

(22) PCT Filed: Oct. 11, 2022
(86) PCT No.: PCT/EP2022/078193

§ 371 Date: Apr. 24, 2024

Related U.S. Application Data
(60) Provisional application No. 63/271,262, filed on Oct. 25, 2021.

(30) Foreign Application Priority Data
Nov. 29, 2021 (EP) 21210975.5

(51) Int. Cl. A61M 5/20

(52) U.S. Cl. CPC A61M 5/2033; A61M 2005/2006

(57) ABSTRACT

The present disclosure generally relates to medicament delivery devices such as autoinjectors, and particularly concerns cover structures configured to surround a medicament delivery member at a proximal end of the medicament delivery device.

### CROSS-REFERENCE TO RELATED APPLICATIONS

The present application is a U.S. National Phase Application pursuant to 35 U.S.C. § 371 of International Application No. PCT/EP2022/078193 filed Oct. 11, 2022, which claims priority to (i) U.S. Provisional Patent Application No. 63/271,262 filed Oct. 25, 2021, and (ii) European Patent Application No. 21210975.5 filed Nov. 29, 2021. The entire disclosure contents of these applications are herewith incorporated by reference into the present application.

### TECHNICAL FIELD

The present disclosure generally relates to medicament delivery devices such as autoinjectors, and particularly concerns cover structures configured to surround a medicament delivery member at a proximal end of the medicament delivery device.

### BACKGROUND

A number of medical conditions require injections. These days, a number of different injection devices exist, including various types of pen injectors, autoinjectors and on-body devices. Although many of these devices have enabled major improvements in the management of a number of medical conditions, various limitations do still exist in the current technology. Not least amongst these are the difficulties faced by patients that require frequent injections and by patients that need to inject particularly viscous drugs. In considering these problems, the applicant has appreciated that various developments could be made to help improve the medicament delivery devices on the market today, which are set out in more detail below.

### SUMMARY

An object of the present disclosure is to provide a cover structure for a medicament delivery device which solves, or at least mitigates problems of the prior art.

According to a first aspect of the present disclosure, there is provided a cover structure for a medicament delivery device. The cover structure is configured to surround a medicament delivery member at a proximal end of the medicament delivery device. The cover structure is movable in an axial direction of the medicament delivery device between a retracted position in which the medicament delivery member is exposed at the proximal end of the cover structure, and an extended position in which the medicament delivery member is covered by the cover structure. The cover structure comprising a lock structure at the proximal end of the cover structure. The lock structure comprising at least one deflectable clip having a distal end and a proximal end, and an actuation surface configured to receive an actuating force. The at least one deflectable clip being arrangeable in a neutral position in which the at least one deflectable clip prevents the cover structure from moving axially from the extended position to the retracted position. The deflectable clip further being arrangeable in a deflected position, reachable upon acting on the actuating surface, in which the cover structure is allowed to move axially towards the retracted position. The deflectable clip is configured so that the deflectable clip is transitioned from the neutral position to the deflected position when an actuating force is applied on the actuating surface starting from the proximal end and moving towards the distal end.

Embodiments of the present disclosure provides for advantageous cover structures that allows for the medicament delivery member to be exposed, so that the medicament delivery device can be activated only when the at least one deflectable clip is pushed into a deflected state. In other words, it can be ensured that the medicament delivery member, e.g., a needle, is not exposed when it is not intended to and thereby potentially cause injury. Further, by means of the lock structure, the user is forced to take appropriate action to use the medicament delivery device. For example, the lock structure may be releasable by a cooperating structure of a medicament delivery assisting device acting on the actuating surface, thereby forcing the user to use the medicament delivery assisting device which advantageously may ensure that the medicament is properly delivered to the user.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the components thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

The same direction terminology has been used to describe other components such as the pad—for example, the proximal end of the pad is the part of the pad closest to the dose delivery site (injection site), and the distal end of the pad is the part of the pad furthest from the dose delivery site. In the Figures, the longitudinal direction is the direction of axis 102, with the corresponding circumferential direction 31 and radial direction 32 relative to the axis 102 also shown.

When the wording ‘at the injection site’ or ‘at the dose delivery site’ is used in this application, it generally refers to the point where the medicament delivery device (e.g. a needle) enters the patient, along with the surrounding area, for example the area where the pad is attached.

According to one embodiment, the at least one deflectable clip may be configured to be transitioned to the deflected state by cooperation with a circumferential structure of an attachment portion of a pad configured to receive the medicament delivery device, acting on the actuating surface. Such pad, when in use, is attached at an injection site of the user. The pad includes the attachment portion that, when in use may lock a medicament delivery device in place, until a user disengages the medicament delivery device. Advantageously, the circumferential structure of the pad acts on the actuating surface when the medicament delivery device is inserted in the attachment portion, whereby the cover structure is transitioned to the retracted position. The deflectable clips are specifically adapted to be deflected by the circumferential structure of the attachment portion. Thus, the user is encouraged to use the pad which provides for ensuring that the medicament is properly delivered, for example assisting the user in maintaining the medicament delivery device in place at the injection site for a sufficient time duration.

According to one embodiment, the circumferential structure may be cylindrical structure, and the lock structure is configured to fit inside the cylindrical structure. A cylindrical structure advantageously provides for rotational symmetry, thus, the circumferential orientation of the cover structure in the circumferential structure is of less or no importance for causing a deflection of the clips.

According to one embodiment, the at least one clip, when being in the neutral position and the cover structure is moved towards the retracted position, may be configured to abut against a stop element of the medicament delivery device. The stop element is preferably distally located with respect to the at least one clip when the cover structure is in a extended position.

According to one embodiment, the stop element may be part of a housing of the medicament delivery device.

According to one embodiment, a distal end of the at least one clip may be configured to abut against the stop element for preventing the cover structure from moving to the retracted position.

According to one embodiment, the deflectable clip may be configured such that when the deflectable clip is deflected transversally inwards, the stop element is released whereby the cover structure is movable towards the retracted position. Accordingly, the deflectable clips are protruding radially outwards in their neutral position.

According to one embodiment, the locking structure may comprise at least two deflectable clips that are symmetrically arranged along a circumference of the cover structure.

According to one embodiment, the at least one deflectable clip may each comprise a ramp surface reaching from a proximal end to a distal end of the clip, and that may be configured to slide on the cylindrical structure of the attachment portion of the pad.

According to one embodiment, wherein the at least one deflectable clip comprises fixed proximal end and a free distal end, the free distal end being movable in relation to the fixed end when the at least one deflectable clip is transitioned from the neutral position to the deflected position.

According to one embodiment, wherein the at least one deflectable clip is made in a cut-through of the cover structure. Hereby, a relatively simple way of providing the cover structure and clips as an integrated single structure is provided.

According to one embodiment, wherein, when in the neutral position and the cover structure is in the extended position, the at least one deflectable clip are positioned proximally outside a housing of the medicament delivery device.

There is further provided a medicament delivery device comprising the cover structure of any of the herein disclosed embodiments.

There is according to a second aspect of the present disclosure provided a pad extending along an axis from a proximal end to a distal end, where the proximal end is the end of the pad adjacent to an injection site when the pad is in use. The pad comprises an attachment portion configured to receive a medicament delivery device, and a circumferential structure arranged in the attachment portion. The circumferential structure being configured to receive and push on a lock structure of a cover structure comprised in the medicament delivery device when in use and the medicament delivery device is moved into place in the attachment portion, so that the locking structure is moved from a neutral position in which the locking structure prevents the cover structure, when in the extended position, from moving axially towards the retracted position, to a deflected position in which the cover structure is allowed to move axially towards the retracted position in the attachment portion.

Hereby, an advantageous pad is provided that includes a circumferential structure able to cooperate with a locking structure of a cover structure as described in the first aspect. This pad ensures that the medicament delivery device is maintained in proper position for the medicament to be delivered. Further, the circumferential structure is specifically adapted for enabling actuation of the lock structure, only when the medicament delivery device is in place in the attachment portion, thereby encouraging the user to use the pad.

According to one embodiment, the proximal injection surface may comprise an adhesive. The adhesive provides for attaching the pad to the injection site.

This aspect provides similar advantages as the ones discussed above with regards to the first aspect.

There is according to a third aspect of the present disclosure provided a cover structure for a medicament delivery device, wherein the cover structure is configured to surround a medicament delivery member at a proximal end of the medicament delivery device. The cover structure is movable in an axial direction of the medicament delivery device between a retracted position in which the medicament delivery member is exposed at the proximal end of the cover structure, and an extended position with the medicament delivery member covered by the cover structure. The cover structure comprises a pad integrated with the cover structure, the pad extending along an axis from the proximal end to a distal end, where the proximal end is the end of the pad adjacent to an injection site when the pad is in use.

Users of medicament delivery devices are encouraged to use a pad for facilitating the task of injecting the medicament, especially ensuring that the medicament delivery device is maintained in place at the injection site for a sufficient time period. Embodiments of the third aspect of the present disclosure provides for a cover structure having an integrated the pad, to ensure that a pad is employed when using the medicament delivery device.

According to one embodiment, a proximal injection surface of the pad may comprise an adhesive.

According to one embodiment, the cover structure may comprise a release mechanism which, upon being actuated, releases the cover structure from the retracted position.

According to one embodiment, the release mechanism may be configured to latch onto locking protrusions of a housing of the medicament delivery device.

According to one embodiment, the cover structure may comprise resilient tabs adapted to apply transversal force on a housing of the medicament delivery device.

According to one embodiment, the pad and the cover structure may be made in a single piece.

According to one embodiment, the cover structure may comprise the medicament delivery member arranged covered by a removable protection cap at the proximal end of the pad.

According to one embodiment, the medicament delivery member may be configured to be fully transitioned to the interior of the cover structure once the removable protection cap is removed.

According to one embodiment, the cover structure may comprise a receiving portion configured to connect the medicament delivery member to a medicament container of a medicament delivery device when the medicament delivery device is attached to the cover structure.

This third aspect provides similar advantages as the ones discussed above with regards to the first aspect and second aspect.

Generally, all terms used in the claims are to be interpreted according to their ordinary meaning in the technical field, unless explicitly defined otherwise herein. All references to “a/an/the member, apparatus, component, means, etc.” are to be interpreted openly as referring to at least one instance of the member, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 is a perspective view of a pad and an autoinjector according to embodiments of the present disclosure;

FIG. 2 is a side view of the pad and autoinjector in FIG. 1;

FIG. 3A is a cross-section of the autoinjector and pad in FIGS. 1 and 2 in a state before the autoinjector is engaged with the pad;

FIG. 3B-C are cross-sections of the autoinjector and pad in FIGS. 1 and 2 as the autoinjector is inserted in an attachment portion of the pad;

FIG. 4A-B are cross-sections of an autoinjector according to embodiments; and

FIG. 5A-D illustrate cross-sections of a further possible embodiment of the present disclosure.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

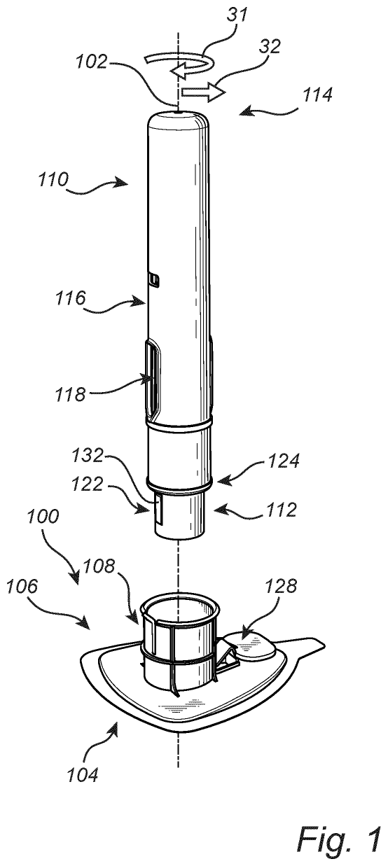

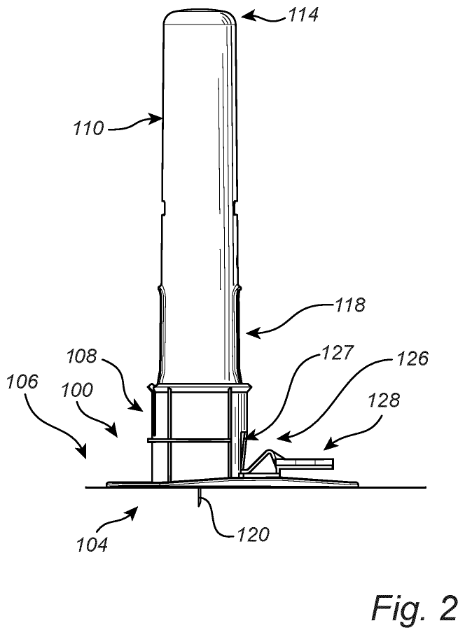

FIGS. 1 and 2 show an example of a medicament delivery device 110 and a pad 100 according to embodiments of the present disclosure. The pad 100 extends along an axis 102 from a proximal end 104 to a distal end 106. The proximal end 104 is the end of the pad adjacent to an injection site when the pad is in use. The injection site is understood as the drug delivery site.

The pad 100 comprises an attachment portion 108 configured to receive a medicament delivery device 110, such as an autoinjector or pen injector.

The medicament delivery device 110 in FIGS. 1 and 2 extends from a proximal end 112 to a distal end 114 relative to the axis 102. The medicament delivery device 110 comprises a housing 116, a window 118, a needle 120, and a cover structure 122 configured to cover the needle 120. The medicament delivery device 110 also comprises an attachment portion 124, which is configured to engage with the attachment portion 108 of the pad 100.

The attachment portion 108 of the pad 100 is equipped with a locking mechanism 126 for releasable locking the medicament delivery device 110 to the pad 100. The locking mechanism 126 may be configured in various ways, for example as a friction lock where a movable arm 127 applies a holding force on the attachment portion 124 of the medicament delivery device 110, or a snap-fit lock, where a protrusion on the proximal end of the housing 116 of the medicament delivery device 110 engages with a corresponding part on the attachment portion 108 of the pad 100, locking the medicament delivery device 110 into place.

Further, the pad 100 optionally comprises a release button 128 configured such that actuating the release button 128 releases the medicament delivery device from the attachment portion 108.

FIG. 2 illustrates the medicament delivery device 110 attached to the pad 100 at the attachment portion 108. The needle 120 extends out from the proximal end 104 of the pad 100. In this state, the cover structure, sometimes referred to as a needle guard, is in a retracted position, thereby exposing the needle 120. It is desirable that the needle 120 is not maintained in this exposed state if the medicament delivery device 110 is not attached in the attachment portion 108. Further, it is desirable that a pad 100 is used when using the medicament delivery device 110 for delivering a medicament at an injection site, thus it is desirable that a user is encouraged, or even forced, to use a pad 100, to ensure proper and safe delivery of the medicament.

For addressing the above problems, the cover structure 122 is provided with a locking structure which will now be described in more detail.

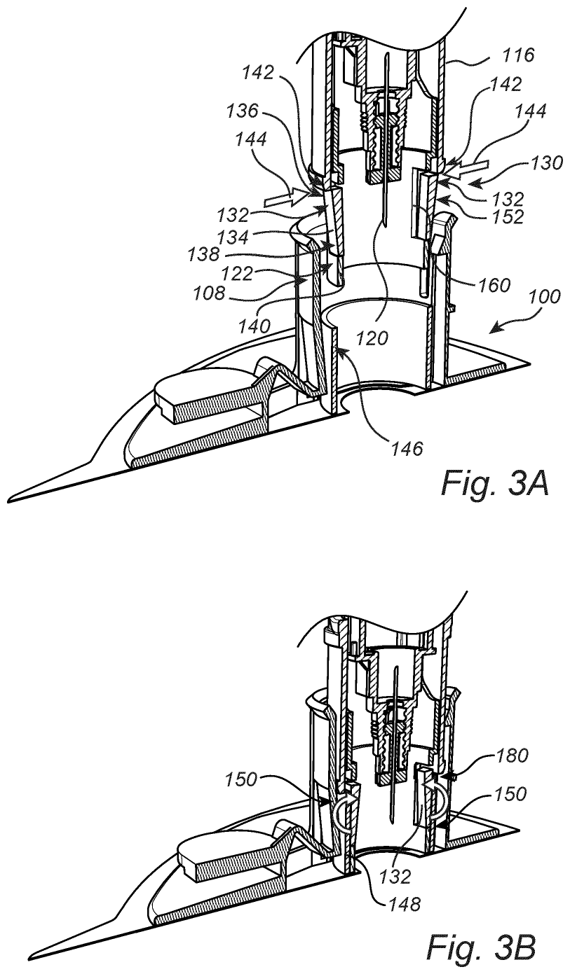

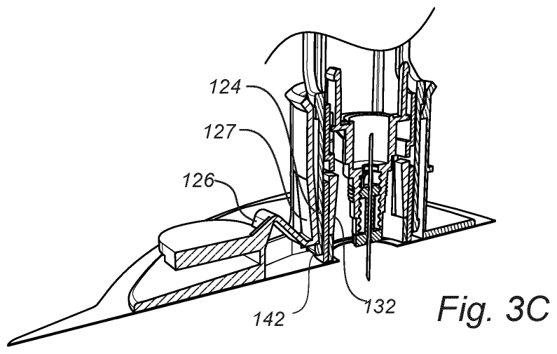

Turning now to FIGS. 3A-3C each showing a cross-section of a cover structure 122 of a medicament delivery device 110. The cover structure 122 is configured to surround a medicament delivery member 120 at a proximal end 112 of the medicament delivery device 110. The general operation of the cover structure is that it is movable in an axial direction, along axis 102, better seen in FIG. 1, of the medicament delivery device 110 between a retracted position in which the medicament delivery member 120 is exposed at the proximal end 112 of the cover structure 122, and an extended position in which the medicament delivery member 120 is covered by the cover structure 122. In the extended position of the cover structure 122, the medicament delivery member 120 is covered such that it is prevented from causing unnecessary injury. The cover structure 122 is more proximally located than the medicament delivery member 120 in the extended position.

The cover structure 122 comprises a lock structure 130 at the proximal end 112 of the cover structure. The lock structure 130 comprising at least one deflectable clip 132 having a distal end 136 and a proximal end 138, and an actuation surface 134 configured to receive an actuating force. The at least one deflectable clip 132 being arrangeable in a neutral position in which the at least one deflectable clip 132 prevents the cover structure 122 from moving axially from the extended position to the retracted position as illustrated in FIG. 3A where the clip 132 abuts against the stop element 140.

The deflectable clips 132 further being arrangeable in a deflected position, illustrated in FIGS. 3B-C, reachable upon acting on the actuating surface 134, so that the cover structure 122 is allowed to move axially towards the retracted position illustrated in FIG. 3C. The clips 132 are configured so that they are transitioned from the neutral position to the deflected position when a actuating force is applied on the actuating surface 134 starting from the proximal end 138 and moving towards the distal end 136. Preferably, a sliding force is applied on the actuating surface starting from the proximal end 138 and sliding towards the distal end 136.

In the neutral position the actuation surface 134 of the deflectable clip 132 incrementally reaches further radially away from a proximal cylindrical portion 140 of the cover structure 122 on the distal end 136 of the clip compared to the proximal end 138 of the clip 132. In the neutral position, the actuating surface 134 is inclined with respect to the longitudinal axis 102 of the medicament delivery device 110.

Turning now specifically to FIG. 3A, the proximal end 112 of the medicament delivery device 110 is shown just before it is inserted in an attachment portion 108 of a pad 100. The needle 120 is covered by or accommodated inside the cover structure 122 which here is in the extended position. The cover extension 122 is slidable inside the housing 116 in the longitudinal direction of the medicament delivery device 110 such that it can move between the extended position and the retracted position. The cover structure 122 may be spring biased towards the extended position.

The cover structure 122 is generally cylindrical with a hollow inner space for housing the needle 120. Further, the clips 132 provide for a tapered shape of a portion of the cover structure 122, with the wider part of the tapered shape being at the distal end 136 of the clips 132.

At the most proximal end of the cover structure 122 a cylindrical portion 140 is located and provides for facilitating correctly fitting the cover structure 122 in the attachment portion 108 of the pad 108. The cylindrical portion 140 is proximally arranged with respect to the clips 132.

In this neutral position of the clips 132, they abut against a stop element 142 of the medicament delivery device 110 to prevent that the cover structure 122 is moved towards the retracted position. In this specific embodiment it is the distal end 136 of the at least one clip 132 that is configured to abut against the stop element 142. Further, the deflectable clip 132 are positioned proximally outside the housing 116 of the medicament delivery device 110.

If the clips 132 are deflected radially inwards, as indicated by arrows 144, the clips are deflected transversally inwards to a location transversally closer to the longitudinally axis 102 than the stop element 142, whereby the cover structure 122 is no longer prevented by the stop element 140 from moving longitudinally towards the retracted position, as will be described in more detail herein.

The pad 100 comprises the attachment portion 108 configured to receive the medicament delivery device 110. More specifically, in use, the cover structure 122 is inserted inside the attachment portion 108, guided by the cylindrical portion 140. The proximal end 112 of the housing 116 comprising the stop element 140 is also fitted inside the attachment portion.

A circumferential structure 146 is arranged in the attachment portion 108. Turning to FIG. 3B, as the medicament delivery device 110 is inserted in the attachment portion 108, the at least one deflectable clip 132 is transitioned to the deflected state by cooperation with the circumferential structure 146 of an attachment portion 108. For this, the circumferential structure 146 acts on the actuating surface 134 to deflect the clips 132 as indicated by arrows 150 . . . . In this example embodiment, the actuating surface 134 faces radially outwards, in a transverse direction. In other words, the circumferential structure 146 acts in a radial direction towards the longitudinal axis 102 of the medicament delivery device 110. The circumferential structure 146 slides on the actuation surface 134 from the proximal end 138 towards distal end 136.

Accordingly, the circumferential structure 146 is configured to receive and push on a clip 132 of the lock structure 130 of the cover structure 122 when the medicament delivery device 110 is moved into place in the attachment portion 108. In this embodiment, the circumferential structure 146 is a cylindrical structure, and the lock structure is configured to fit inside the cylindrical structure.

As discussed above, as a result, the locking structure is moved from a neutral position in which the locking structure prevents the cover structure 122, when in the extended position, from moving axially towards the retracted position, to a deflected position in which the cover structure 122 is allowed to move axially towards the retracted position in the attachment portion.

In FIG. 3B, the cover structure 122 is still in the extended position, and with the clips 132 in the deflected position. As the cover structure 122 is pushed towards the proximal support 148 of the attachment portion 108, adjacent to the injection site when the pad 100 and medicament delivery device 110 is in use, the cover extension 122, is allowed to move in the longitudinal direction towards the extended position, shown in FIG. 3C. The stop element 142 is moved to a space transversally outside the circumferential structure 146. Here, the movable arm 127 of the locking mechanism 126 applies a holding force on the attachment portion 124 of the medicament delivery device 110,

The clips are preferably resilient. Thus, the clips 132 spring back to the neutral position once released by the stop element 142.

The locking structure 130 preferably comprises at least two deflectable clips 132 that are symmetrically arranged along a circumference of the cover structure 122.

In order to facilitate for the deflecting motion of the clips 132, the clips 132 each comprise a ramp surface 152 reaching from the proximal end 138 to the distal end 136, and that is configured to slide on the cylindrical structure of the attachment portion of the pad. This ramp surface 138 may be the entire actuation surface 134, or the actuation surface 134 may comprise the ramp surface 152.

The proximal end 138 of the clip is here a fixed end about which the clip can rotate as indicated by arrows. For this, the distal end 136 is a free end that can move in relation to the fixed end when the at least one deflectable clip 132 is transitioned from the neutral position to the deflected position.

On way to realise the clips 132 is to make them in a cut-through of the cover structure. Thus, the clips 132 and the cover structure 122 are made in a one integral part, by cutting through the material of the cover structure 122 to form the clips. The cutting may be made in e.g., a U-shape.

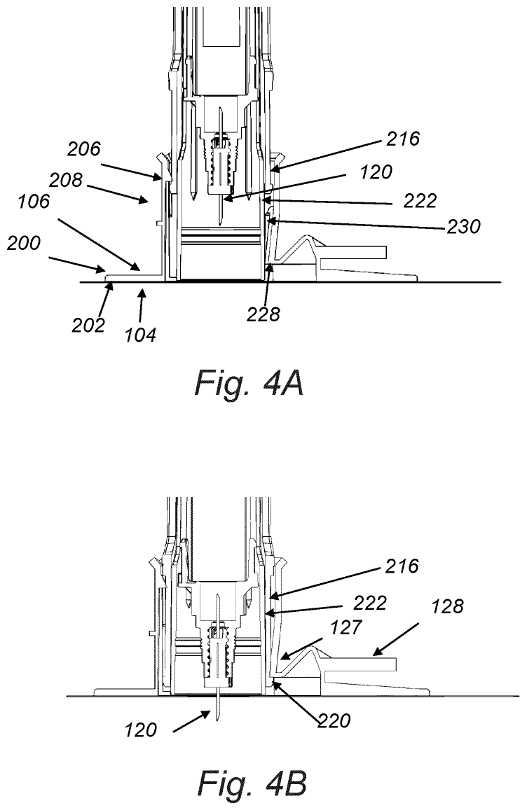

FIG. 4A-B illustrate a further aspect of the present disclosure related to a cover structure 222 for a medicament delivery device 110. As in the first aspect, the cover structure 222 is configured to surround a medicament delivery member 120 at the proximal end 112 of the medicament delivery device 110.

Further, also in this embodiment, the cover structure 222 is movable in an axial direction of the medicament delivery device 210 between a retracted position, shown in FIG. 4B, in which the medicament delivery member 120 is exposed at the proximal end 112 of the cover structure, and an extended position, shown in FIG. 4A, with the medicament delivery member 120 covered by the cover structure 222.

The cover structure 222 comprises a pad 200 integrated with the cover structure 222. As with the pad 100 discussed in relation to preceding drawings, the pad 200 extends along an axis 102 from a proximal end 104 to a distal end 106, where the proximal end 104 is the end of the pad 200 adjacent to an injection site when the pad is in use.

The cover structure 222 is a cylindrical structure with a hollow inner space for accommodating the medicament delivery member 120. The cover structure 222 may thus have tubular body.

The cover structure 222 is arranged coaxially with the outer cylindrical wall 206 of the attachment portion 208 of the pad 200, extending proximally from the distal end 106 of the pad. The cover structure 222 is located inside the outer cylindrical wall 206 of the attachment portion 208, thereby having a smaller diameter than the outer cylindrical wall 206.

Further, the cover structure 222 is fixedly attached to the pad 200, for example to the attachment portion 208. Here, the cover structure 222 reaches to the proximal end 104 of the pad 200.

In FIG. 4A, the cover structure 222 is in an extended position with the needle 120 covered. As the medicament delivery device is moved towards the injection site, in a proximal direction, the cover structure 222 is moved longitudinally in a distal direction so that the needle 120 is exposed on the proximal side 104 of the pad, see FIG. 4B. In this retracted position, the housing 216 of the medicament delivery device 210 has moved into the space between the cover structure 222 and the outer cylindrical wall 206 of the attachment portion 208. In other words, in this retracted position, the housing 216, the cover structure 222, and the outer cylindrical wall 206 are coaxially oriented with respect to each other.

Preferably, the pad 200 and the cover structure 222 are made in a single piece. For example, the pad 200 with the attachment portion 208 may be molded together with the cover structure 222. The cover structure 222 is part of the medicament delivery device 110.

Preferably, a proximal injection surface 202 of the pad comprises an adhesive. The proximal injection surface 202 is in contact with the injection site when the medicament delivery device 210 is in use.

The cover structure 222, which includes the integral pad 200 and attachment portion 208 here further comprises a locking mechanism 126 which, upon being actuated, releases the cover structure 222 from the retracted position.

The locking mechanism 126 may be configured in various ways, for example as a friction lock where a movable arm 127 applies a holding force on the housing 216 of the medicament delivery device 210, or a snap-fit lock, where a protrusion on the proximal end of the housing 216 of the medicament delivery device 210 engages with a corresponding part on the attachment portion 208 of the pad 200, locking the cover structure 222 in the retracted position.

Further, the pad 100 optionally comprises a release button 128 configured such that actuating the release button 128 releases the housing 216, and thereby the cover structure 222 from the retracted position.

The housing 216 comprises a locking protrusion 220 at the proximal end 112 of the housing. The protrusions 220 extending in a transversal direction away from the longitudinal axis 102 and latches onto the proximal end shoulder 228 of the release mechanism movable arm 127. When the release button is pushed, the shoulder 228 moves away from the housing 216 and releases the protrusion 220.

Further, to improve the locking of the housing 216 to thereby maintain the cover structure 22s in the retracted position, a set of resilient tabs 230 attached to the cover structure, in the space between the cover structure 222 and the outer wall 206, are adapted to apply transversal force on the housing 216 of the medicament delivery device.

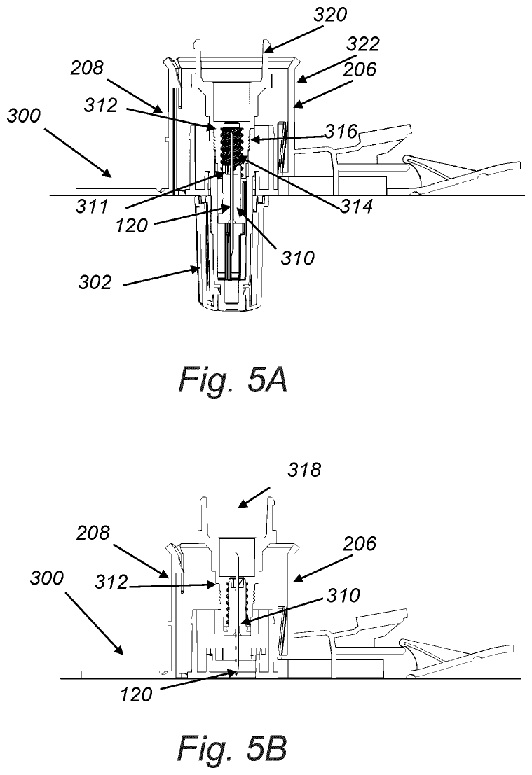

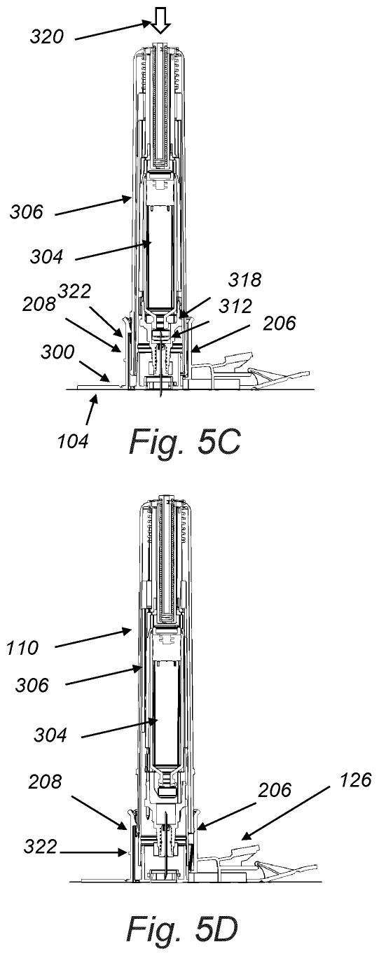

FIG. 5A-D illustrates a cross-section of one possible embodiment of the present disclosure in which the medicament delivery device 110 has a medicament container 304, e.g. a cartridge, in its main body 306, and where the pad 100 comprises the medicament delivery member, the needle 120.

The pad 100 comprises an attachment portion 208 as discussed in relation to other embodiments described herein. However, in this embodiment, the attachment portion itself, more specifically the cylindrical wall 206 serve as cover structure. However, the cover structure is not initially part of the medicament delivery device, but, as will be discussed below, once in use, the main body of the medicament delivery device 110 is attached to a needle assembly 308 accommodated in the attachment portion 208.

The needle 120 is arranged in through-hole of a needle hub 310 having a tubular shape. The needle 120 extends beyond the proximal end 104 of the pad 100 and is arranged covered by a removable protection cap 302.

FIG. 5B illustrates the needle 120 being fully transitioned to the interior of the cover structure 322 once the removable protection cap 302 is removed. The transition of the needle can be realized in various ways. In one possible implementation, when the cap 302 is turned, the needle hub 310 is also turned due to a structural engagement therebetween.

For example, a distal engagement member 311 of the hub 310 is a threaded member designed to interact with the threads 314 of a central passage of the retainer member 312. Threads 316 between the cap 302 and the retainer member 312 have a different direction of the pitch than the threads 314 between the hub 310 and the retainer member 312. Therefore, when the cap 302 is turned for removing it from the retainer member 312 in the proximal direction, the turning of the hub 310 causes it to move in the distal direction into the retainer member 312. It should be understood that this turning type needle isolation protection is only shown as an example and other possible ways to realize that the needle is moved into the attachment portion 208 are possible.

The retainer member 312 comprises a tubular receiving portion 318 configured to received and connect the needle 120 to the medicament container 304 of the medicament delivery device 110 when it is attached to the cover structure 322, being the cylindrical wall 206. As shown in FIG. 5C, the main body 306 of the medicament delivery device is pushed into the retainer member 312, as indicated by the arrow 320, whereby the needle 120 protrudes out from the proximal end 104 of the pad 100. Thus, the cover structure 322 is in a retracted position.

FIG. 5D illustrates the medicament delivery device 110 after use, with the needle being covered by the cover structure 322, here being in its extended position. After use, the user can eject the medicament delivery device 100 using the locking mechanism 126, and dispose the medicament delivery device 100 and the pad 300.

The pad described herein may be made out of transparent material. Further, the sidewall of the attachment portion may comprise an opening forming a window close to the base of the attachment portion adjacent the pad surface. Prior to use, instructions tell the patient to see if the cover extension is fully depressed when using the auto injector. With the window, the patient is able to inspect the status of the cover extension when using the autoinjector in combination with the injection support pad.

The pads disclosed herein may include embedded electronics which includes different units.

A sensor unit which may recognize injection events, such as the autoinjector inserted into the attachment portion, injection started, and injection ends.

A memory unit which is configured to store the recorded data during the injection.

A connectivity unit configured to transmit the stored data to a smart device or the network directly.

A processing unit configured to control the entire system and processes the data before transmitting it.

User interface units that are configured to provide feedback to the patient, such as status LEDs, haptic, and/or audio feedback.

When the auto-injector is placed into the attachment portion, the sensors inside of the support pad are configured to recognize the event and give feedback to the patient via haptic/visual or audio elements.

When the injection finishes, the sensors are configured to recognize the event and give feedback to the patient again. Further, the collected data is stored in the memory unit and may be transmitted to the smart device/network via the connectivity unit after the injection event finishes.

The sensor can be one of or the combination of the following: a mechanical switch, a Hall-effect sensor, an accelerometer

The mechanical switch, hall-effect sensor, or accelerometer can be used for detection of the insertion of the auto-injector into the injection port.

The accelerometer can be used for detecting injection events.

Possible wireless communication methods include Bluetooth and Cellular Networks.

Bluetooth connectivity requires a smart device to transmit the stored data to the network and it requires a pairing action between the support pad and the smart device before being able to use the supporting pad. But it's a cheaper alternative and it requires less space on PCB.

The cellular network does not require any pairing process, it can be used as a plug-n-play device, no prior setup is needed, but it's more expensive and it requires more space on PCB.

Depending on the requirements of the product any of those two technologies can be used.

Such processing units may comprise a logic circuit or control unit including a microprocessor, microcontroller, programmable digital signal processor or another programmable device. The processing circuitry may also, or instead, each include an application specific integrated circuit, a programmable gate array or programmable array logic, a programmable logic device, or a digital signal processor. Where the processing circuitry includes a programmable device such as the microprocessor, microcontroller or programmable digital signal processor mentioned above, the processor may further include computer executable code that controls operation of the programmable device.

The inventive concept has mainly been described above with reference to a few examples. However, as is readily appreciated by a person skilled in the art, other embodiments than the ones disclosed above are equally possible within the scope of the inventive concept, as defined by the appended claims.

Some aspects are described in the following clauses.

1. A cover structure (222, 322) for a medicament delivery device (210), wherein the cover structure is configured to surround a medicament delivery member (120) at a proximal end (112) of the medicament delivery device,
the cover structure is movable in an axial direction of the medicament delivery device between a retracted position in which the medicament delivery member is exposed at the proximal end of the cover structure, and an extended position with the medicament delivery member covered by the cover structure, the cover structure comprising:
a pad (200) integrated with the cover structure, the pad extending along an axis (102) from a proximal end (104) to a distal end (106), where the proximal end is the end of the pad adjacent to an injection site when the pad is in use.

2. The cover structure according to clause 1, wherein a proximal injection surface (202) of the pad comprises an adhesive.

3. The cover structure according to any one of clauses 1 and 2, comprising a locking mechanism (126) which, upon being actuated, releases the cover structure from the retracted position.

4. The cover structure according to clause 3, wherein the locking mechanism (126) is configured to latch onto a locking protrusion (220) of a housing of the medicament delivery device.

5. The cover structure according to any one of clauses 1 to 4, comprising resilient tabs (230) adapted to apply transversal force on a housing (216) of the medicament delivery device.

6. The cover structure according to any one of clauses 1 to 5, wherein the pad and the cover structure are made in a single piece.

7. The cover structure according to any one of clauses 1 to 5, wherein the cover structure is part of the medicament delivery device.

8. The cover structure (322) according to any one of clauses 1 to 6, comprising the medicament delivery member arranged covered by a removable protection cap (302) at the proximal end of the pad.

9. The cover structure according to clause 8, the medicament delivery member being configured to be fully transitioned to the interior of the cover structure once the removable protection cap is removed.

10. The cover structure according to any one of clauses 8 and 9, comprising a receiving portion (318) configured to connect the medicament delivery member to a medicament container (304) of a medicament delivery device when the medicament delivery device is attached to the cover structure.

11. The cover structure according to any one of clauses 8 to 10, wherein a main body (306) of the medicament delivery device comprising a medicament container is inserted in the cover structure when in use.

1-15. (canceled)

16. A cover structure for a medicament delivery device, the cover structure is configured to surround a medicament delivery member at a proximal end of the medicament delivery device,
the cover structure is movable in an axial direction of the medicament delivery device between a retracted position in which the medicament delivery member is exposed at the proximal end of the cover structure, and an extended position in which the medicament delivery member is covered by the cover structure,
the cover structure comprising a lock structure at the proximal end of the cover structure, the lock structure comprising at least one deflectable clip having a distal end and a proximal end, and an actuation surface configured to receive an actuating force, the at least one deflectable clip being arrangeable in a neutral position in which the at least one deflectable clip prevents the cover structure from moving axially from the extended position to the retracted position, the deflectable clip further being arrangeable in a deflected position, reachable upon acting on the actuating surface, in which the cover structure is allowed to move axially towards the retracted position, wherein the deflectable clip is configured so that the deflectable clip is transitioned from the neutral position to the deflected position when a actuating force is applied on the actuating surface starting from the proximal end and moving towards the distal end.

17. The cover structure according to claim 16, the at least one deflectable clip being configured to be transitioned to the deflected state by cooperation with a circumferential structure of an attachment portion of a pad configured to receive the medicament delivery device, acting on the actuating surface.

18. The cover structure according to claim 16, wherein the circumferential structure is a cylindrical structure, and the lock structure is configured to fit inside the cylindrical structure.

19. The cover structure according to claim 16, wherein the at least one clip, when being in the neutral position and the cover structure is moved towards the retracted position, is configured to abut against a stop element of the medicament delivery device.

20. The cover structure according to claim 19, wherein the stop element is part of a housing of the medicament delivery device.

21. The cover structure according to claim 19, wherein the distal end of the at least one clip is configured to abut against the stop element.

22. The cover structure according to claim 16, wherein the deflectable clip is configured such that when the deflectable clip is deflected transversally inwards, the stop element is released whereby the cover structure is movable towards the retracted position.

23. The cover structure according to claim 16, comprising at least two deflectable clips that are symmetrically arranged along a circumference of the cover structure.

24. The cover structure according to claim 16, wherein the at least one deflectable clip each comprises a ramp surface reaching from a proximal end to a distal end, and that is configured to slide on the cylindrical structure of the attachment portion of the pad.

25. The cover structure according to claim 16, wherein the at least one deflectable clip comprises fixed proximal end and a free distal end, the free distal end being movable in relation to the fixed end when the at least one deflectable clip is transitioned from the neutral position to the deflected position.

26. The cover structure according to claim 16, wherein the at least one deflectable clip is made in a cut-through of the cover structure.

27. The cover structure according to claim 16, wherein, when in the neutral position and the cover structure is in the extended position, the at least one deflectable clip are positioned proximally outside a housing of the medicament delivery device.

28. A medicament delivery device comprising the cover structure of claim 16.

29. A pad extending along an axis from a proximal end to a distal end, where the proximal end is the end of the pad adjacent to an injection site when the pad is in use, the pad comprising:
an attachment portion configured to receive a medicament delivery device, and
a circumferential structure arranged in the attachment portion, the circumferential structure being configured to receive and push on a lock structure of a cover structure comprised in the medicament delivery device when in use and the medicament delivery device is moved into place in the attachment portion, so that the locking structure is moved from a neutral position in which the locking structure prevents the cover structure, when in the extended position, from moving axially towards the retracted position, to a deflected position in which the cover structure is allowed to move axially towards the retracted position in the attachment portion.

30. A cover structure for a medicament delivery device, wherein the cover structure is configured to surround a medicament delivery member at a proximal end of the medicament delivery device,
the cover structure is movable in an axial direction of the medicament delivery device between a retracted position in which the medicament delivery member is exposed at the proximal end of the cover structure, and an extended position with the medicament delivery member covered by the cover structure, the cover structure comprising:
a pad integrated with the cover structure, the pad extending along an axis from a proximal end to a distal end, where the proximal end is the end of the pad adjacent to an injection site when the pad is in use.
